SEQUENCE LISTING (3 sequences)

SEQ ID NO: 1 - DNA, 21 bp, Artificial Sequence
  Synthetic sense siRNA
gugaucuucaccgacaagatt

SEQ ID NO: 2 - DNA, 21 bp, Artificial Sequence
  Synthetic antisense siRNA
ucuugucggugaagaucactt

SEQ ID NO: 3 - DNA, 4733 bp, Artificial Sequence
  pEGFP-N1
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

(19) United States
(12) Patent Application Publication
(10) Pub. No.: US 2012/0093762 A1
(43) Pub. Date: Apr. 19, 2012

(54) NUCLEIC ACID DELIVERY COMPOUNDS

(75) Inventors: Padmanabh Chivakula, San Diego, CA (US); Jian Liu, San Diego, CA (US); Sang Van, San Diego, CA (US); Akinobu Soma, Carlsbad, CA (US); Keisaku Okada, San Diego, CA (US); Lei Yu, Carlsbad, CA (US); Nianchun Ma, Oceanside, CA (US)

(73) Assignee: Nitto Denko Corporation, Osaka (JP)

(21) Appl. No.: 13/273,100

(22) Filed: Oct. 13, 2011

Related U.S. Application Data
(60) Provisional application No. 61/393,297, filed on Oct. 14, 2010.
(60) Provisional application No. 61/454,922, filed on Mar. 21, 2011.

(51) Int. Cl. A61K 47/34; A61P 35/00; C12N 5/071; C08G 69/48

(52) U.S. Cl. 424/78.17; 525/426; 525/54.2; 435/375

(57) ABSTRACT

Polymers including two or more different recurring units are disclosed herein. Also disclosed herein are methods of using such polymers to deliver nucleic acids to a cell.

### RELATED APPLICATION INFORMATION

This application claims priority to U.S. Provisional Application Ser. No. 61/393,297, filed on Oct. 14, 2010, and U.S. Provisional Application Ser. No. 61/454,922, filed on Mar. 21, 2011, which are incorporated herein by reference in their entireties for all purposes.

### SEQUENCE LISTING

The present application is being filed along with a Sequence Listing in electronic format. The Sequence Listing is provided as a file entitled KUZU1_{—}017AUS_SequenceListing.TXT, created Oct. 7, 2011, which is 6.41 kb in size. The information in the electronic format of the Sequence Listing is incorporated herein by reference in its entirety.

### BACKGROUND

1. Field

Disclosed herein are compositions and methods related to the fields of organic chemistry, pharmaceutical chemistry, biochemistry, molecular biology, and medicine. More specifically, embodiments described herein relate to compounds, compositions, and methods using a compound and/or composition described herein for delivering a nucleic acid into a cell.

2. Description

A number of techniques are available for delivering a nucleic acid such as siRNA into a cell, including the use of viral transfection systems and non-viral transfection systems. Non-viral transfection systems can include, for example, polymers, lipids, liposomes, micelles, dendrimers, and nanomaterials. Examples of polymers that have previously been studied for cell transfection include some cationic polymers.

Each type of system has its respective advantages and drawbacks. For example, viral systems can yield high transfection efficiency, but may not be as safe as some non-viral systems. (See Verma I M et al. Nature (1997) 389: 239-242; Marshall E. Science (2000) 286: 2244-2245). In addition, viral systems can be complicated and/or expensive to prepare. Non-viral transfection systems, such as cationic polymers, have been reported to transfer plasmid DNA into cells. Some drawbacks to the use of some cationic polymers include their toxicity to cells and/or their lack of stability.

### SUMMARY

Some embodiments disclosed herein relate to a polymer that can include a first recurring unit of Formula (I) and a second recurring unit of Formula (II).

Some embodiments disclosed herein relate to a polymer that can include a first recurring unit of Formula (I), a second recurring unit of Formula (II) and a third recurring unit of Formula (VII).

In some embodiments, a polymer described herein can be associated with a nucleic acid. For example, the nucleic acid can be selected from DNA, RNA siRNA and antisense.

Some embodiments described herein relate to a pharmaceutical composition that can include a polymer described herein and a pharmaceutically acceptable carrier. In some embodiments, the pharmaceutical composition can further include a nucleic acid.

Some embodiments described herein relate to a method of transfecting a cell that can include delivering to a cell a polymer described herein associated with a nucleic acid. Other embodiments described herein relate to using a polymer described herein associated with a nucleic acid in the preparation of a medicament for transfecting a cell. Still other embodiments described herein relate to a polymer described herein associated with a nucleic acid for transfecting a cell.

Some embodiments described herein relate to a method of treating a tumor that can include administering an effective amount of a polymer described herein associated with a nucleic acid. Other embodiments described herein relate to using a polymer described herein associated with a nucleic acid in the preparation of a medicament for treating a tumor. Still other embodiments described herein relate to a polymer described herein associated with a nucleic acid for treating a tumor. Some embodiments described herein relate to a method of treating a tumor that can include contacting a tumor cell with an effective amount of a polymer described herein associated with a nucleic acid.

### BRIEF DESCRIPTION OF THE DRAWINGS

FIG. 1 is a schematic illustrating an example of a polymer with a group having a pH transition point.

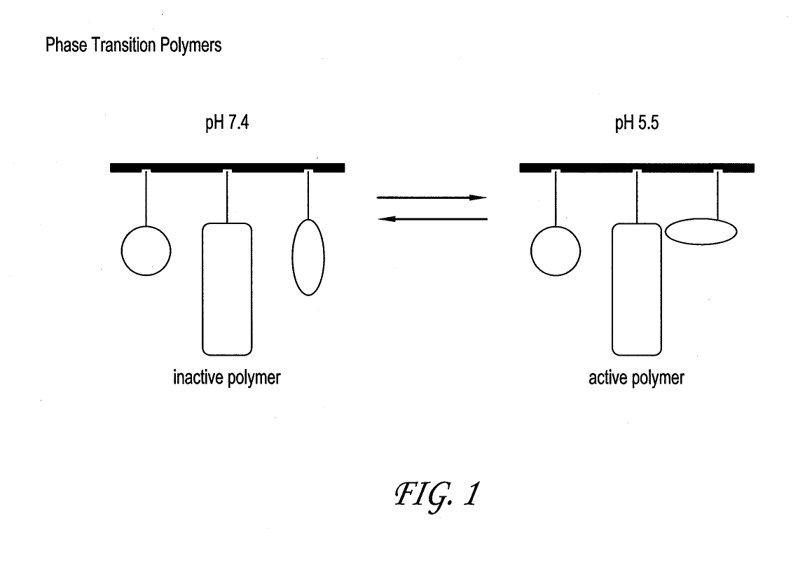

FIG. 2 depicts a reaction scheme for the synthesis of a polymer that includes a first recurring unit of Formula (I), a second recurring unit of Formula (II), and a third recurring unit of Formula (VII) that includes a group that has a pH transition point.

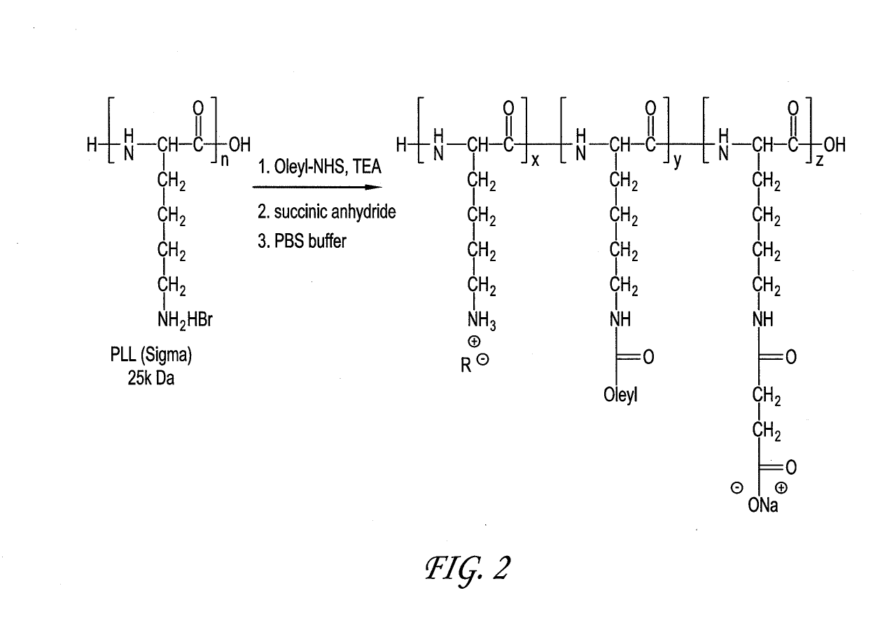

FIG. 3 illustrates one example of a change in chemical properties that can occur in a polymer at a pH transition point.

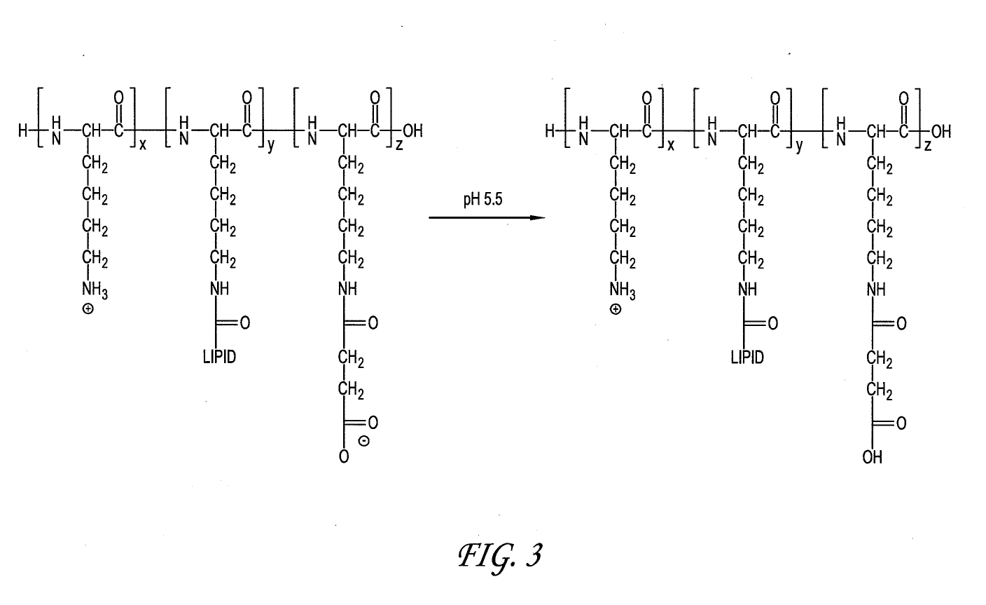

FIG. 4 depicts DNA (gene) transfection efficiency for several polymers.

FIG. 5 illustrates a bar graph depicting the results of a cell viability assay for several polymers.

FIGS. 6A-C illustrate the results of a gel retardation assay for several polymers described herein that are associated with siRNA.

FIG. 7 is a ¹H NMR spectrum of a polymer synthesized as described herein.

### DETAILED DESCRIPTION

Unless defined otherwise, all technical and scientific terms used herein have the same meaning as is commonly understood by one of ordinary skill in the art. All patents, applications, published applications and other publications referenced herein are incorporated by reference in their entirety, unless stated otherwise. In the event that there are a plurality of definitions for a term herein, those in this section prevail unless stated otherwise.

As used herein, the term “pH transition point” refers to the pH above or below which a group having a pH transition point experiences a change in one or more chemical properties. Examples of such chemical properties include, but are not limited to, hydrophilicity, lipophilicity, solubility, polarity, and electric charge.

Where at least two molecules are “associated” it means that the molecules are in electronic interaction with each other. Such interaction may take the form of a chemical bond, including, but not limited to, a covalent bond, a polar covalent bond, an ionic bond, an electrostatic bond, a coordinate covalent bond, an aromatic bond, a hydrogen bond, a dipole, or a van der Waals interaction. Those of ordinary skill in the art understand that the relative strengths of such interactions may vary widely.

As used herein, “Cₘ to Cₙ” in which “m” and “n” are integers refers to the number of carbon atoms in an alkyl or alkenyl group. That is, the alkyl or alkenyl can contain from “m” to “n”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “m” and “n” are designated with regard to an alkyl or alkenyl group, the broadest range described in these definitions is to be assumed.

As used herein, “alkyl” refers to a straight or branched hydrocarbon chain fully saturated (no double or triple bonds) hydrocarbon group. An alkyl group can be substituted or unsubstituted.

As used herein, “alkenyl” refers to an alkyl group that contains in the straight or branched hydrocarbon chain one or more double bonds. An alkenyl group can be substituted or unsubstituted.

Whenever a group is described as being “optionally substituted” that group may be unsubstituted or substituted with one or more of the indicated substituents. Likewise, when a group is described as being “unsubstituted or substituted” if substituted, the substituent may be selected from one or more the indicated substituents.

Unless otherwise indicated, when a substituent is deemed to be “optionally substituted,” or “substituted” it is meant that the substituent is a group that may be substituted with one or more group(s) individually and independently selected from C₁-C₁₂ alkyl, C₂-C₁₂ alkenyl, C₂-C₁₂ alkynyl, C₃-C₁₂ cycloalkyl, C₃-C₁₂ cycloalkenyl, C₃-C₁₂ cycloalkynyl, C₅-C₁₂ aryl, heteroaryl (containing 5-12 atoms, wherein 1-3 atoms are heteroatoms selected from O, N, and S, and the remaining atoms are carbon), heteroalicyclyl (containing 5-12 atoms, wherein 1-3 atoms are heteroatoms selected from O, N, and S, and the remaining atoms are carbon), C₄-C₂₄ aralkyl, heteroaralkyl (containing 6-24 atoms, wherein 1-3 atoms are heteroatoms selected from O, N, and S, and the remaining atoms are carbon), (heteroalicyclyl)alkyl (containing 6-24 atoms, wherein 1-3 atoms are heteroatoms selected from O, N, and S, and the remaining atoms are carbon), hydroxy, alkoxy, aryloxy, acyl (—C═OR, wherein R is an optionally substituted alkyl, an optionally substituted cycloalkyl, or an optionally substituted aryl), ester, mercapto, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, sulfenyl, sulfonyl, sulfonyl, haloalkyl (mono-, di- and tri-substituted haloalkyl), haloalkoxy (mono-, di- and tri-substituted haloalkoxy), and amino, including mono- and di-substituted amino groups.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure or be stereoisomeric mixtures. In addition it is understood that, in any compound having one or more double bond(s) generating geometrical isomers that can be defined as E or Z each double bond may independently be E or Z or a mixture thereof. Likewise, all tautomeric forms are also intended to be included.

As used herein, the abbreviations for any protective groups, amino acids and other compounds are, unless indicated otherwise, in accord with their common usage, recognized abbreviations, or the IUPAC-IUP Commission on Biochemical Nomenclature (See, Biochem. 11:942-944 (1972)).

Some embodiments herein are directed a polymer that can include a first recurring unit of Formula (I) and a second recurring unit of Formula (II), wherein Formulae (I) and (II) can each have the following structures:

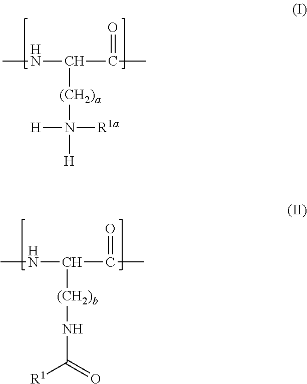

In Formulae (I) and (II), each a and each b can independently be 1, 2, 3, 4, 5, or 6; R^{1a} can be absent or H, and if R^{1a} is H, the nitrogen atom to which R^{1a} is attached can have an associated positive charge; and R¹ can be selected from an optionally substituted C₄-C₂₄ alkyl, an optionally substituted C₄-C₂₄ alkenyl and an optionally substituted steryl. In some embodiments, R¹ can be an optionally substituted substituent selected from oleyl, lauryl, myristyl, palmityl, margaryl, stearyl, arachidyl, behenyl, lignoceryl and steryl. In other embodiments, R¹ can be an optionally substituted C₆-C₁₈ alkyl, an optionally substituted C₁₅-C₂₀ alkyl, an optionally substituted C₄-C₂₀ alkyl, or an optionally substituted C₁₅-C₂₄ alkyl. In yet other embodiments, R¹ can be an optionally substituted C₆-C₁₈ alkenyl, an optionally substituted C₁₅-C₂₀ alkenyl, an optionally substituted C₄-C₂₀ alkenyl, or an optionally substituted C₁₅-C₂₄ alkenyl. In one embodiment, R¹ can be a C₁₇ alkenyl. For example, R¹ can be —(CH₂)₇CH═CH(CH₂)₇CH₃. In another embodiment, R¹ can be a C₈ alkyl. In yet another embodiment, R¹ can be an optionally substituted cholesterol. Advantageously, in some embodiments, R¹ can be a lipophilic group. In some embodiments, each a and/or each b can be 2, 3, 4, or 5. In other embodiments, each a and/or each b can be 3, 4, or 5. In some embodiments, each a and/or each b can be 4. In some embodiments, R^{1a} can be absent. In some embodiments, R^{1a} can be H, and the nitrogen atom to which R^{1a} is attached can have an associated positive charge.

In some embodiments, the polymer can further include a recurring unit of Formula (VII):

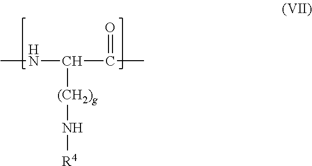

In Formula (VII), each g can independently be 1, 2, 3, 4, 5, or 6; and R⁴ can include a group that has a pH transition point. In some embodiments, g can be 2, 3, 4, or 5. In other embodiments, g can be 3, 4, or 5. In some embodiments, g can be 4.

R⁴ can include a group that has a pH transition point. Above or below the pH transition point, the R⁴ group can experience a change in one or more chemical properties. In some embodiments, R⁴ can be hydrophilic at a pH that is greater than or equal to the pH transition point. In other embodiments, R⁴ can be hydrophobic at a pH that is less than the pH transition point. In one embodiment, R⁴ can be hydrophilic at a pH that is greater than or equal to the pH transition point and hydrophobic at a pH that is less than the pH transition point.

The position of the pH transition point can be controlled by selecting the appropriate chemical structure for R⁴. In some embodiments, the transition point can be at a pH<7.4 (e.g., less than physiological pH and/or less than the pH of blood). In other embodiments, the transition point can be at a pH<6. In yet other embodiments, the transition point can be at a pH<5. In some embodiments, the transition point can be at a pH that is generally equal to the pH of the micro-environment of tumor tissue (e.g., in or substantially adjacent to tumor tissue). Those skilled in the art may appreciate that the micro-environment of tumor tissue can be acidic relative to physiological pH. Advantageously, in some embodiments, R⁴ can be hydrophilic in a generally physiological environment and can be generally hydrophobic at or substantially adjacent to tumor tissue.

A wide variety of groups having a pH transition point can be used. In some embodiments, a group having a pH transition point can be directly bonded to the NH group of the third recurring unit of Formula (VII). In other embodiments, a group having a pH transition point can be bonded to the NH group of the third recurring unit of Formula (VII) through a linking group. Examples of linking groups include, but are not limited to, low molecular weight linking groups comprising 1-12 atoms, such as acrylate, alkylene, amide, amine, ester, carbonate, carbonyl, ether, thioamide and combinations thereof, and high molecular weight linking groups such as polyethylene glycol (PEG). Examples of groups having a pH transition point include, but are not limited to, amines (cyclic and aliphatic; amino, mono-substituted amines, and di-substituted amines), morpholine and carboxylates. In some embodiments, a group having a pH transition point, for example, R⁴, can be a group that includes an imidazolyl group, a

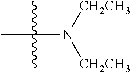

group, a

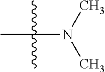

group or a morpholinyl group. In some embodiments, R⁴ can have the following structure: —C(═O)—(CH₂)₁₋₄—C(═O)OR^{A} at a pH≧the transition point, wherein R^{A} can be an alkali metal, such as sodium. In some embodiments, R⁴ can have the following structure: —C(═O)—(CH₂)₂—C(═O)OR^{A} at a pH≧the transition point, wherein R^{A} can be an alkali metal, such as sodium. In other embodiments, R⁴ can have the following structure:

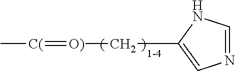

at a pH≧the transition point. In still other embodiments, R⁴ can have the following structure:

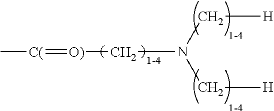

at a pH≧the transition point. In yet still other embodiments, R⁴ can have the following structure:

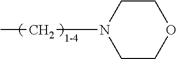

at a pH≧the transition point. In some embodiments, R⁴ can have the following structure:

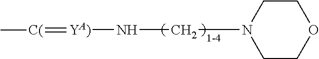

at a pH≧the transition point, wherein Y^{A} can be S or O.

In some embodiments, the polymer can include the following structure, wherein m and n can each independently be a positive integer in the range of about 1 to about 2000. In the following structure, a first recurring unit of Formula (I) can be connected to a second recurring unit of Formula (II) and/or another first recurring unit of Formula (I). Also, a second recurring unit of Formula (II) can be connected to a first recurring unit of Formula (I) and/or another second recurring unit of Formula (II).

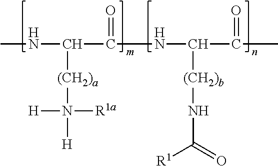

In some embodiments, the polymer can include the following structure, wherein m, n, and o can each independently be a positive integer in the range of about 1 to about 2000. In the following structure, a first recurring unit of Formula (I) can be connected to a second recurring unit of Formula (II), a third recurring unit of Formula (VII), and/or another first recurring unit of Formula (I). Similarly, a second recurring unit of Formula (II) can be connected to a first recurring unit of Formula (I), a third recurring unit of Formula (VII), and/or another second recurring unit of Formula (II); and a third recurring unit of Formula (VII) can be connected to a first recurring unit of Formula (I), a second recurring unit of Formula (II), and/or another third recurring unit of Formula (VII).

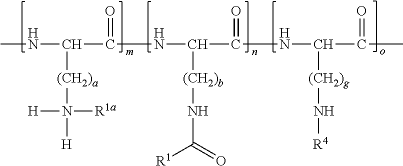

The polymers described herein can also include a nucleic acid. The nucleic acid can be associated with the polymer in a variety of ways. In some embodiments, the nucleic acid can be associated with the polymer via an electrostatic bond. In some embodiments, the nucleic acid can be selected from DNA, RNA, siRNA, and antisense. In one embodiment, the nucleic acid can be siRNA. In some embodiments, the nucleic acid, such as siRNA, can be associated with at least one recurring unit of Formula (I). For example, the nucleic acid can be associated with the terminal NH₂R^{1a} group of at least one recurring unit of Formula (I). In some embodiments, the nucleic acid can have a biological effect upon a cell to which it is delivered, in vitro, ex vivo and/or in vivo. For example, those skilled in the art will appreciate that reference herein to the use of a polymer for therapeutic and/or treatment purposes may refer to the use of the polymer in combination with a nucleic acid with which it is associated.

In some embodiments, the nucleic acid can be associated with the polymer via a covalent bond. In some embodiments where the nucleic acid and polymer are associated via a covalent bond, the nucleic acid can be directly covalently bonded to the polymer. In other embodiments, the nucleic acid can be indirectly bonded to the polymer through a linking group. Examples of linking groups include, but are not limited to, low molecular weight linking groups comprising 1-12 atoms, such as acrylate, alkylene, amide, amine, ester, carbonate, carbonyl, ether, thioamide and combinations thereof, and high molecular weight linking groups such as polyethylene glycol (PEG). In some embodiments, the polymer can be covalently bonded to a thiol-reactive agent, including but not limited to iodoacetamide, maleimide, benzylic halide, bromomethylketone, and orthopyridyldisulfide reagent. In some embodiments, the thiol-reactive agent can be covalently bonded to the terminal NH₂R^{1a} group of at least one recurring unit of Formula (I) through a PEG linking group. In one embodiment, the linking group can be —(CH₂CH₂O)ₙ—, wherein n is an integer in the range of from about 1 to about 25. In some embodiments, n can be an integer in the range of from about 1 to 5. In other embodiments, n can be an integer in the range of from about 12 to about 24. In one embodiment, n can be 4. In some embodiments, a nucleic acid can be modified to include a thiol group according to methods known to those skilled in the art. In some embodiments, the thiol group on the nucleic acid can be covalently bonded directly to the thiol-reactive group on the polymer. In some embodiments, the nucleic acid can be selected from DNA, RNA, siRNA, and antisense. In some embodiments, the nucleic acid can be siRNA. In some embodiments, the nucleic acid can have a biological effect upon a cell to which it is delivered, in vitro, ex vivo and/or in vivo. For example, those skilled in the art will appreciate that reference herein to the use of a polymer for therapeutic or treatment purposes may refer to the use of the polymer in combination with the nucleic acid with which it is associated.

Some embodiments herein are directed to a polymer that can include a first recurring unit of Formula (I) and a second recurring unit of Formula (II). Other embodiments are directed to a polymer that can include a first recurring unit of Formula (I), a second recurring unit of Formula (II), and a third recurring unit of Formula (VII). The relative amounts of the first recurring unit, the second recurring unit, and the third recurring unit present in the polymer can vary widely.

In some embodiments, the polymer can include ≧5% mole percent of the recurring unit of Formula (I) based on the ratio of total moles of recurring units of Formula (I) to the total moles of recurring units in the polymer. In some embodiments, the polymer can include ≧10% mole percent of the recurring unit of Formula (I) based on the ratio of total moles of recurring units of Formula (I) to the total moles of recurring units in the polymer. In some embodiments, the polymer can include ≧30% mole percent of the recurring unit of Formula (I) based on the ratio of total moles of recurring units of Formula (I) to the total moles of recurring units in the polymer. In some embodiments, the polymer can include ≧50% mole percent of the recurring unit of Formula (I) based on the ratio of total moles of recurring units of Formula (I) to the total moles of recurring units in the polymer. In some embodiments, the polymer can include ≧90% mole percent of the recurring unit of Formula (I) based on the ratio of total moles of recurring units of Formula (I) to the total moles of recurring units in the polymer.

In some embodiments, the polymer can include a total amount of the recurring unit of Formula (I) in the range of about 50 mole % to about 70 mole % based on the ratio of total moles of recurring units of Formula (I) to the total moles of recurring units in the polymer. In some embodiments, the polymer can include a total amount of the recurring unit of Formula (I) in the range of about 10 mole % to about 60 mole % based on the ratio of total moles of recurring units of Formula (I) to the total moles of recurring units in the polymer. In some embodiments, the polymer can include a total amount of the recurring unit of Formula (I) in the range of about 20 mole % to about 50 mole % based on the ratio of total moles of recurring units of Formula (I) to the total moles of recurring units in the polymer. In yet another embodiment, the polymer can include a total amount of the recurring unit of Formula (I) in the range of about 70 mole % to about 95 mole % based on the ratio of total moles of recurring units of Formula (I) to the total moles of recurring units in the polymer. In some embodiments, the polymer can include a total amount of the recurring unit of Formula (I) of about 10 mole %, about 20 mole %, about 30 mole %, about 40 mole %, about 50 mole %, about 60 mole %, about 70 mole %, about 80 mole %, or about 90 mole % based on the ratio of total moles of recurring units of Formula (I) to the total moles of recurring units in the polymer.

In some embodiments, the polymer can include at least about 50 recurring units of Formula (I). In other embodiments, the polymer can include at least about 100 recurring units of Formula (I). In other embodiments, the polymer can include at least about 200 recurring units of Formula (I). In other embodiments, the polymer can include at least about 500 recurring units of Formula (I). In other embodiments, the polymer can include at least about 1000 recurring units of Formula (I). In other embodiments, the polymer can include at least about 1500 recurring units of Formula (I). In other embodiments, the polymer can include about 2000 recurring units of Formula (I).

In some embodiments, the polymer can include from about 50 to about 2000 recurring units of Formula (I). In other embodiments, the polymer can include from about 200 to about 1500 recurring units of Formula (I). In yet other embodiments, the polymer can include from about 300 to about 700 recurring units of Formula (I). In other embodiments, the polymer can include from about 50 to about 100 recurring units, from about 1500 to about 2000 recurring units, from about 1000 to about 1500 recurring units, or from about 700 to about 1000 recurring units of Formula (I). In yet other embodiments, the polymer can include from about 100 to about 200, from about 100 to about 500, or from about 300 to about 600 recurring units of Formula (I).

In some embodiments, the polymer can include ≧5% mole percent of the recurring unit of Formula (II) based on the ratio of total moles of recurring units of Formula (II) to the total moles of recurring units in the polymer. In some embodiments, the polymer can include ≧10% mole percent of the recurring unit of Formula (II) based on the ratio of total moles of recurring units of Formula (II) to the total moles of recurring units in the polymer. In some embodiments, the polymer can include ≧20% mole percent of the recurring unit of Formula (II) based on the ratio of total moles of recurring units of Formula (II) to the total moles of recurring units in the polymer. In some embodiments, the polymer can include ≧30% mole percent of the recurring unit of Formula (II) based on the ratio of total moles of recurring units of Formula (II) to the total moles of recurring units in the polymer. In some embodiments, the polymer can include ≧50% mole percent of the recurring unit of Formula (II) based on the ratio of total moles of recurring units of Formula (II) to the total moles of recurring units in the polymer. In some embodiments, the polymer can include ≧70% mole percent of the recurring unit of Formula (II) based on the ratio of total moles of recurring units of Formula (II) to the total moles of recurring units in the polymer.

In some embodiments, the polymer can include a total amount of the recurring unit of Formula (II) in the range of about 5 mole % to about 70 mole % based on the ratio of total moles of recurring units of Formula (II) to the total moles of recurring units in the polymer. In some embodiments, the polymer can include a total amount of the recurring unit of Formula (II) in the range of about 10 mole % to about 60 mole % based on the ratio of total moles of recurring units of Formula (II) to the total moles of recurring units in the polymer. In some embodiments, the polymer can include a total amount of the recurring unit of Formula (II) in the range of about 20 mole % to about 50 mole % based on the ratio of total moles of recurring units of Formula (II) to the total moles of recurring units in the polymer. In some embodiments, the polymer can include a total amount of the recurring unit of Formula (II) in the range of about 1 mole % to about 40 mole % based on the ratio of total moles of recurring units of Formula (II) to the total moles of recurring units in the polymer. In some embodiments, the polymer can include a total amount of the recurring unit of Formula (II) of about 1 mole %, about 5 mole %, about 10 mole %, about 20 mole %, about 30 mole %, about 40 mole %, about 50 mole %, about 60 mole %, or about 70 mole % based on the ratio of total moles of recurring units of Formula (II) to the total moles of recurring units in the polymer.

In some embodiments, the polymer can include at least about 50 recurring units of Formula (II). In other embodiments, the polymer can include at least about 100 recurring units of Formula (II). In other embodiments, the polymer can include at least about 200 recurring units of Formula (II). In other embodiments, the polymer can include at least about 500 recurring units of Formula (II). In other embodiments, the polymer can include at least about 1000 recurring units of Formula (II). In other embodiments, the polymer can include at least about 1500 recurring units of Formula (II). In other embodiments, the polymer can include about 2000 recurring units of Formula (II).

In some embodiments, the polymer can include from about 50 to about 2000 recurring units of Formula (II). In other embodiments, the polymer can include from about 200 to about 1500 recurring units of Formula (II). In yet other embodiments, the polymer can include from about 300 to about 700 recurring units of Formula (II). In other embodiments, the polymer can include from about 50 to about 100 recurring units, from about 1500 to about 2000 recurring units, from about 1000 to about 1500 recurring units, or from about 700 to about 1000 recurring units of Formula (II). In yet other embodiments, the polymer can include from about 100 to about 200, from about 100 to about 500, or from about 300 to about 600 recurring units of Formula (II).

In some embodiments, the polymer can include ≧1% mole percent of the recurring unit of Formula (VII) based on the ratio of total moles of recurring units of Formula (VII) to the total moles of recurring units in the polymer. In some embodiments, the polymer can include ≧5% mole percent of the recurring unit of Formula (VII) based on the ratio of total moles of recurring units of Formula (VII) to the total moles of recurring units in the polymer. In some embodiments, the polymer can include ≧10% mole percent of the recurring unit of Formula (VII) based on the ratio of total moles of recurring units of Formula (VII) to the total moles of recurring units in the polymer. In some embodiments, the polymer can include ≧30% mole percent of the recurring unit of Formula (VII) based on the ratio of total moles of recurring units of Formula (VII) to the total moles of recurring units in the polymer. In some embodiments, the polymer can include ≧60% mole percent of the recurring unit of Formula (VII) based on the ratio of total moles of recurring units of Formula (VII) to the total moles of recurring units in the polymer. In some embodiments, the polymer may not include any recurring units of Formula (VII).

In some embodiments, the polymer can include a total amount of the recurring unit of Formula (VII) in the range of about 1 mole % to about 60 mole % based on the ratio of total moles of recurring units of Formula (VII) to the total moles of recurring units in the polymer. In some embodiments, the polymer can include a total amount of the recurring unit of Formula (VII) in the range of about 5 mole % to about 50 mole % based on the ratio of total moles of recurring units of Formula (VII) to the total moles of recurring units in the polymer. In some embodiments, the polymer can include a total amount of the recurring unit of Formula (VII) in the range of about 10 mole % to about 40 mole % based on the ratio of total moles of recurring units of Formula (VII) to the total moles of recurring units in the polymer. In other embodiments, the polymer can include a total amount of the recurring unit of Formula (VII) in the range of from about 20 mole % to about 30 mole % based on the ratio of total moles of recurring units of Formula (VII) to the total moles of recurring units in the polymer. In some embodiments, the polymer can include a total amount of the recurring unit of Formula (VII) of about 1 mole %, about 5 mole %, about 10 mole %, about 20 mole %, about 25 mole %, about 30 mole %, about 40 mole %, about 50 mole %, or about 60 mole % based on the ratio of total moles of recurring units of Formula (VII) to the total moles of recurring units in the polymer.

In some embodiments, the polymer can include at least about 5 recurring units of Formula (VII). In other embodiments, the polymer can include at least about 10 recurring units of Formula (VII). In other embodiments, the polymer can include at least about 50 recurring units of Formula (VII). In other embodiments, the polymer can include at least about 100 recurring units of Formula (VII). In other embodiments, the polymer can include at least about 200 recurring units of Formula (VII). In other embodiments, the polymer can include at least about 500 recurring units of Formula (VII). In other embodiments, the polymer can include at least about 1000 recurring units of Formula (VII). In other embodiments, the polymer can include at least about 1500 recurring units of Formula (VII). In other embodiments, the polymer can include about 2000 recurring units of Formula (VII).

In some embodiments, the polymer can include from about 2 to about 2000 recurring units of Formula (VII). In other embodiments, the polymer can include from about 50 to about 1000 recurring units of Formula (VII). In other embodiments, the polymer can include from about 200 to about 1500 recurring units of Formula (VII). In yet other embodiments, the polymer can include from about 300 to about 700 recurring units of Formula (VII). In other embodiments, the polymer can include from about 50 to about 100 recurring units, from about 1500 to about 2000 recurring units, from about 1000 to about 1500 recurring units, or from about 700 to about 1000 recurring units of Formula (VII). In yet other embodiments, the polymer can include from about 2 to about 100, from about 5 to about 80, from about 10 to about 50, from about 100 to about 200, from about 100 to about 500, or from about 300 to about 600 recurring units of Formula (VII).

In some embodiments directed to a polymer that includes a recurring unit of Formula (I) and a recurring unit of Formula (II), a majority of the recurring units in the polymer can be recurring units of Formulae (I) and/or (II). In some embodiments directed to a polymer that includes a recurring unit of Formula (I) and a recurring unit of Formula (II), at least 50 mole % of the total moles of recurring units in the polymer can be recurring units of Formulae (I) and (II) (e.g., the sum of the moles of recurring units of Formula (I) and moles of recurring units of Formula (II) in the polymer can be equal to at least 50 mole % of the total moles of recurring units in the polymer). In some embodiments directed to a polymer that includes a recurring unit of Formula (I) and a recurring unit of Formula (II), at least 75 mole % of the total moles of recurring units in the polymer can be recurring units of Formulae (I) and (II). In some embodiments directed to a polymer that includes a recurring unit of Formula (I) and a recurring unit of Formula (II), at least 85 mole % of the total moles of recurring units in the polymer can be recurring units of Formulae (I) and (II). In some embodiments directed to a polymer that includes a recurring unit of Formula (I) and a recurring unit of Formula (II), at least 95 mole % of the total moles of recurring units in the polymer can be recurring units of Formulae (I) and (II). In some embodiments directed to a polymer that includes a recurring unit of Formula (I) and a recurring unit of Formula (II), at least 98 mole % of the total moles of recurring units in the polymer can be recurring units of Formulae (I) and (II). In some embodiments directed to a polymer that includes a recurring unit of Formula (I) and a recurring unit of Formula (II), at least 99 mole % of the total moles of recurring units in the polymer can be recurring units of Formulae (I) and (II). In some embodiments directed to a polymer that includes a recurring unit of Formula (I) and a recurring unit of Formula (II), about 100 mole % of the total moles of recurring units in the polymer can be recurring units of Formulae (I) and (II) (e.g., the polymer does not include any recurring units other than the recurring units of Formulae (I) and (II)).

In some embodiments directed to a polymer that includes a recurring unit of Formula (I) and a recurring unit of Formula (II), the polymer can include a total amount of the recurring units of Formulae (I) and (II) in the range of about 50 mole % to about 99 mole % based on the ratio of total moles of recurring units of Formulae (I) and (II) to the total moles of recurring units in the polymer (e.g., the sum of the moles of recurring units of Formula (I) and moles of the recurring units of Formula (II) in the polymer can be in the range of about 50 mole % to about 99 mole % based on the total moles of recurring units in the polymer). In some embodiments directed to a polymer that includes a recurring unit of Formula (I) and a recurring unit of Formula (II), the polymer can include a total amount of the recurring units of Formulae (I) and (II) in the range of about 70 mole % to about 98 mole % based on the ratio of total moles of recurring units of Formulae (I) and (II) to the total moles of recurring units in the polymer. In some embodiments directed to a polymer that includes a recurring unit of Formula (I) and a recurring unit of Formula (II), the polymer can include a total amount of the recurring units of Formulae (I) and (II) in the range of about 80 mole % to about 95 mole % based on the ratio of total moles of recurring units of Formulae (I) and (II) to the total moles of recurring units in the polymer. In some embodiments directed to a polymer that includes a recurring unit of Formula (I) and a recurring unit of Formula (II), the polymer can include a total amount of the recurring units of Formulae (I) and (II) in the range of about 90 mole % to about 99 mole % based on the ratio of total moles of recurring units of Formulae (I) and (II) to the total moles of recurring units in the polymer. In some embodiments directed to a polymer that includes a recurring unit of Formula (I) and a recurring unit of Formula (II), the polymer can include a total amount of the recurring units of Formulae (I) and (II) of about 50 mole %, about 60 mole %, about 70 mole %, about 80 mole %, about 90 mole %, about 95 mole %, about 98 mole %, about 99 mole % or about 100 mole % based on the ratio of total moles of recurring units of Formulae (I) and (II) to the total moles of recurring units in the polymer.

In some embodiments directed to a polymer that includes a recurring unit of Formula (I) and a recurring unit of Formula (II), the polymer can include from about 50 mole % to about 99 mole % of recurring units of Formula (I), based on the ratio of total moles of recurring units of Formula (I) to the total moles of recurring units in the polymer; from about 1 mole % to about 50 mole % of recurring units of Formula (II), based on the ratio of total moles of recurring units of Formula (II) to the total moles of recurring units in the polymer; and wherein about 95 mole % to about 99 mole % of the total moles of recurring units in the polymer are recurring units of Formulae (I) or (II). In other embodiments directed to a polymer that includes a recurring unit of Formula (I) and a recurring unit of Formula (II), the polymer can include from about 70 mole % to about 95 mole % of recurring units of Formula (I), based on the ratio of total moles of recurring units of Formula (I) to the total moles of recurring units in the polymer; from about 5 mole % to about 30 mole % of recurring units of Formula (II), based on the ratio of total moles of recurring units of Formula (II) to the total moles of recurring units in the polymer; and wherein about 95 mole % to about 99 mole % of the total moles of recurring units in the polymer are recurring units of Formulae (I) or (II).

In some embodiments directed to a polymer that includes a recurring unit of Formula (I), a recurring unit of Formula (II), and a recurring unit of Formula (VII), at least 50 mole % of the total moles of recurring units in the polymer can be recurring units of Formulae (I), (II), and (VII) (e.g., the sum of the moles of recurring units of Formula (I), moles of recurring units of Formula (II), and moles of recurring units of Formula (VII) in the polymer can be equal to at least 50 mole % of the total moles of recurring units in the polymer). In some embodiments directed to a polymer that includes a recurring unit of Formula (I), a recurring unit of Formula (II), and a recurring unit of Formula (VII), at least 75 mole % of the total moles of recurring units in the polymer can be recurring units of Formulae (I), (II), and (VII). In some embodiments directed to a polymer that includes a recurring unit of Formula (I), a recurring unit of Formula (II), and a recurring unit of Formula (VII), at least 85 mole % of the total moles of recurring units in the polymer can be recurring units of Formulae (I), (II), and (VII). In some embodiments directed to a polymer that includes a recurring unit of Formula (I), a recurring unit of Formula (II), and a recurring unit of Formula (VII), at least 95 mole % of the total moles of recurring units in the polymer can be recurring units of Formulae (I), (II), and (VII). In some embodiments directed to a polymer that includes a recurring unit of Formula (I), a recurring unit of Formula (II), and a recurring unit of Formula (VII), at least 98 mole % of the total moles of recurring units in the polymer can be recurring units of Formulae (I), (II), and (VII). In some embodiments directed to a polymer that includes a recurring unit of Formula (I), a recurring unit of Formula (II), and a recurring unit of Formula (VII), at least 99 mole % of the total moles of recurring units in the polymer can be recurring units of Formulae (I), (II), and (VII). In some embodiments directed to a polymer that includes a recurring unit of Formula (I), a recurring unit of Formula (II), and a recurring unit of Formula (VII), about 100 mole % of the total moles of recurring units in the polymer can be recurring units of Formulae (I), (II), and (VII) (e.g., the polymer may not include any recurring units other than the recurring units of Formulae (I), (II), and (VII)).

In some embodiments directed to a polymer that includes a recurring unit of Formula (I), a recurring unit of Formula (II), and a recurring unit of Formula (VII), the polymer can include a total amount of the recurring units of Formulae (I), (II), and (VII) in the range of from about 50 mole % to about 99 mole % based on the ratio of total moles of recurring units of Formulae (I), (II), and (VII) to the total moles of recurring units in the polymer (e.g., the sum of the moles of recurring units of Formula (I), moles of recurring units of Formula (II), and moles of recurring units of Formula (VII) in the polymer can be in the range of from about 50 mole % to about 99 mole % based on the total moles of recurring units in the polymer). In some embodiments directed to a polymer that includes a recurring unit of Formula (I), a recurring unit of Formula (II), and a recurring unit of Formula (VII), the polymer can include a total amount of the recurring units of Formulae (I), (II), and (VII) in the range of about 70 mole % to about 98 mole % based on the ratio of total moles of recurring units of Formulae (I), (II), and (VII) to the total moles of recurring units in the polymer. In some embodiments directed to a polymer that includes a recurring unit of Formula (I), a recurring unit of Formula (II), and a recurring unit of Formula (VII), the polymer can include a total amount of the recurring units of Formulae (I), (II), and (VII) in the range of about 80 mole % to about 95 mole % based on the ratio of total moles of recurring units of Formulae (I), (II), and (VII) to the total moles of recurring units in the polymer. In some embodiments directed to a polymer that includes a recurring unit of Formula (I), a recurring unit of Formula (II), and a recurring unit of Formula (VII), the polymer can include a total amount of the recurring units of Formulae (I), (II), and (VII) in the range of about 90 mole % to about 99 mole % based on the ratio of total moles of recurring units of Formulae (I), (II), and (VII) to the total moles of recurring units in the polymer. In some embodiments directed to a polymer that includes a recurring unit of Formula (I), a recurring unit of Formula (II), and a recurring unit of Formula (VII), the polymer can include a total amount of the recurring units of (I), (II), and (VII) of about 50 mole %, about 60 mole %, about 70 mole %, about 80 mole %, about 90 mole %, about 95 mole %, about 98 mole %, about 99 mole % or about 100 mole % based on the ratio of total moles of recurring units of Formulae (I), (II), and (VII) to the total moles of recurring units in the polymer.

In some embodiments directed to a polymer that includes a recurring unit of Formula (I), a recurring unit of Formula (II), and a recurring unit of Formula (VII), the polymer can include from about 10 mole % to about 85 mole % of recurring units of Formula (I), based on the ratio of total moles of recurring units of Formula (I) to the total moles of recurring units in the polymer; from about 1 mole % to about 10 mole % of recurring units of Formula (II), based on the ratio of total moles of recurring units of Formula (II) to the total moles of recurring units in the polymer; and from about 10 mole % to about 85 mole % of recurring units of Formula (VII), based on the ratio of total moles of recurring units of Formula (VII) to the total moles of recurring units in the polymer; and wherein about 95 mole % to about 99 mole % of the total moles of recurring units in the polymer are recurring units of Formulae (I), (II), or (VII). In other embodiments directed to a polymer that includes a recurring unit of Formula (I) a recurring unit of Formula (II), and a recurring unit of Formula (VII), the polymer can include from about 45 mole % to about 70 mole % of recurring units of Formula (I), based on the ratio of total moles of recurring units of Formula (I) to the total moles of recurring units in the polymer; from about 1 mole % to about 10 mole % of recurring units of Formula (II), based on the ratio of total moles of recurring units of Formula (II) to the total moles of recurring units in the polymer; and from about 25 mole % to about 50 mole % of recurring units of Formula (VII), based on the ratio of total moles of recurring units of Formula (VII) to the total moles of recurring units in the polymer; and wherein about 95 mole % to about 99 mole % of the total moles of recurring units in the polymer are recurring units of Formulae (I), (II), or (VII).

In some embodiments, the polymer can include at least about 55 total recurring units. In other embodiments, the polymer can include at least about 100 total recurring units. In other embodiments, the polymer can include at least about 200 total recurring units. In other embodiments, the polymer can include at least about 500 total recurring units. In other embodiments, the polymer can include at least about 1000 total recurring units. In other embodiments, the polymer can include at least about 1500 total recurring units. In other embodiments, the polymer can include at least about 2000 total recurring units. In other embodiments, the polymer can include at least about 3000 total recurring units. In other embodiments, the polymer can include at least about 4000 total recurring units. In other embodiments, the polymer can include at least about 5000 recurring units.

In some embodiments, the polymer can include from about 55 to about 5000 total recurring units. In other embodiments, the polymer can include from about 100 to about 4000 total recurring units. In yet other embodiments, the polymer can include about 300 to about 700 recurring units. In other embodiments, the polymer can include from about 50 to about 100 recurring units, from about 1500 to about 2000 recurring units, from about 1000 to about 1500 recurring units, or from about 700 to about 1000 recurring units. In yet other embodiments, the polymer can include from about 100 to about 200, from about 100 to about 500, or from about 300 to about 600 recurring units. In still other embodiments, the polymer can include from about 1000 to about 5000, from about 2000 to about 5000, from about 3000 to about 5000, from about 4000 to about 5000, from about 2000 to about 4000, or from about 3000 to about 4000 total recurring units.

In some embodiments, the polymer can have a weight average molecular weight (M_{w}) in the range of from about 8 kDa to about 200 kDa. In other embodiments, the polymer can have a weight average molecular weight in the range of from about 8 kDa to about 150 kDa. In other embodiments, the polymer can have a weight average molecular weight in the range of from about 8 kDa to about 100 kDa. In other embodiments, the polymer can have a weight average molecular weight in the range of from about 10 kDa to about 50 kDa. In other embodiments, the polymer can have a weight average molecular weight in the range of from about 20 kDa to about 45 kDa.

Those skilled in the art also appreciate that the relative mole percentages of the recurring units of Formulae (I), (II), and/or (VII) can be varied depending on the particular recurring units and/or nucleic acid being incorporated into the polymer. Those skilled in the art also appreciate that varying the mole percentages of recurring units of Formulae (I), (II), and/or (VII) relative to the total number of moles of recurring units in the polymer can affect one or more properties of the overall polymer. Examples of these charateristics include, but are not limited to, solubility, degradability, transfection efficiency, and toxicity. In addition, a combination of different recurring units of Formulae (I), (II), and/or (VII) and/or additional recurring units having other structures and/or properties may be included in the polymers described herein depending on the desired characteristics of the polymer. The additional recurring units may be selected based on information available to those skilled in the art as guided by the teachings provided herein.

For example, in some embodiments, the polymer can include the recurring unit of Formula (I) in an amount that allows a nucleic acid to be associated with the polymer (e.g., via an electrostatic or covalent bond) at a weight ratio selected from at least 1:50 (e.g., at least 1 g of nucleic acid to 50 g of polymer), 1:40, 1:30, 1:20, 1:10, 1:5, 1:2.5 and 1.1. The amount of polymer relative to the amount of nucleic acid may be expressed as an amino nitrogen to phosphate ratio (N/P), where N represents the number of terminal NH₂R^{1a} groups from the polymer and P represents the number of phosphate groups from the nucleic acid. In some embodiments, the polymer can include a recurring unit of Formula (I) in an amount that allows the nucleic acid to be associated with the polymer (e.g., via an electrostatic or covalent bond) at a N/P ratio selected from at least 1:1 (e.g., at least 1 NH₂R^{1a} group from the polymer to 1 phosphate group from the nucleic acid), 4:1, 8:1, 12:1, 16:1, and 20:1.

In other embodiments, the polymer can include the recurring unit of Formula (II) in an amount that is effective to give the polymer sufficient lipophilicity to provide a desired degree of cell wall penetration (e.g., disruption of the endosomal membrane) and/or phagocytosis. In yet another example, the polymer can include the recurring unit of Formula (II) in an amount that provides an increased degree of cell wall penetration (as measured, e.g., via a cell transfection assay) as compared to an otherwise similar polymer lacking a recurring unit of Formula (II) (e.g., a polymer including recurring units of Formulae (I) and (VII), but not recurring units of Formula (II)).

As described herein, the recurring unit of Formula (VII) can include a group that has a pH transition point which can affect one or more properties of the overall polymer. Those skilled in the art may appreciate that hydrophilicity and/or hydrophobicity of R⁴ can affect the solubility of the polymer in a particular solvent. Thus, in some embodiments, the polymer can include an amount of the recurring unit of Formula (VII) that is sufficient to affect and/or alter the solubility of the overall polymer.

For example, the polymer can include an amount of the recurring unit of Formula (VII) that makes the polymer relatively less soluble at a first pH and relatively more soluble at a second pH. In some embodiments, an amount of the polymer can be more insoluble in a solvent at a pH less than the transition point compared to the same amount of the same polymer in the same solvent at a pH greater than or equal to the transition point. The solubility can be measured according to any indicia known to those skilled in the art, such as turbidity. The solvent can be any solvent, such as blood or other aqueous solvents.

As described herein, in some embodiments, the transition point can be a pH that is generally equal to the pH of the micro-environment of tumor tissue. In these embodiments, the group having a pH transition point (e.g., a recurring unit of Formula (VII)) can be hydrophilic at a pH generally greater than or equal to the pH transition point and hydrophobic at a pH generally less than the pH transition point. Advantageously, in some embodiments, the polymer can include an amount of the recurring unit of Formula (VII) that makes the polymer more soluble in blood that is outside the micro-environment of tumor tissue, and more insoluble in blood that is within the micro-environment of tumor tissue. In these embodiments, when the polymer is more hydrophobic and/or insoluble in blood within the micro-environment of tumor tissue, it can be more likely to disrupt an endosomal membrane and penetrate a cell. Accordingly, some polymers described herein can selectively penetrate cells and/or deliver nucleic acids at a pH above or below the transition point. Advantageously, some polymers described herein can include an amount of the recurring unit of Formula (VII) that makes the polymer capable of selectively targeting tumor tissue.

As described above, a nucleic acid can be associated with the polymer. Nucleic acids can be commercially available and/or designed according to methods known to those skilled in the art. Various methods for associating the nucleic acid with a polymer described herein can be used. Association of the nucleic acid with the polymer may be carried out, for example, in an aqueous solution or on a solid support, according to methods known to those of ordinary skill in the art as guided by the teachings provided herein.

As described above, a nucleic acid can be covalently bonded to the polymer, e.g., through a linking group. In some embodiments, a polymer that includes a recurring unit of Formula (I) can be intermixed with a moiety that forms the linking group. In some embodiments, the linking group can be covalently bonded with the NH₂R^{1a} group of Formula (I). In some embodiments, the moiety that forms the linking group can include PEG. In one embodiment, the moiety that forms the linking group can be PEG covalently bonded to a thiol-reactive agent, including but not limited to iodoacetamide, maleimide, benzylic halide, bromomethylketone, and orthopyridyldisulfide agents. In some embodiments, the thiol-reactive reagent can be covalently bonded to the NH₂R^{1a} group of Formula (I) through a PEG linking group.

In some embodiments, a nucleic acid can further be intermixed with the polymer. In some embodiments, the nucleic acid can be modified to include a thiol moiety according to methods known to those skilled in the art. In some embodiments, the nucleic acid can be covalently bonded to the recurring unit of Formula (I) and the linking group through the thiol-reactive agent (e.g., a maleimide agent). In other embodiments, the nucleic acid can replace the thiol-reactive agent (e.g., an iodoacetamide, benzylic halide, bromomethylketone, or orthopyridyldisulfide agent) to be covalently bonded to the linking group.

The nucleic acid may be releasable from the polymer. Advantageously, in some embodiments that include an orthopyridyldisulfide agent, the siRNA may advantageously be released from the polymer under reducing conditions. In some embodiments that include another thiol-reactive agent (e.g., an iodoacetamide, maleimide, benzylic halide, or bromomethylketone), the siRNA may not be released from the polymer under reducing conditions, but may be released into its surroundings as the polymer degrades.

One or more of the recurring units (e.g., the first, second, and/or third recurring unit) and/or the nucleic acid can be oriented at various positions relative to the polymer. Such positions may be fixed (e.g., at the middle, ends, or side chains of the polymer) or relative, e.g., the polymer may exhibit a configuration in a particular medium (such as an aqueous medium) such that it has interior and exterior portions.

In some embodiments, one or more recurring units of Formula (I) may be oriented at or near the interior of the polymer. In other embodiments, one or more recurring units of Formula (I) may be oriented at or near the exterior of the polymer. In yet other embodiments, one or more recurring units of Formula (II) may be oriented at or near the exterior of the polymer. In still other embodiments, one or more recurring units of Formula (II) may be oriented at or near the interior of the polymer. In some embodiments, one or more recurring units of Formula (I) can be oriented at or near the interior of the polymer and one or more recurring units of Formula (II) can be oriented at or near the exterior of the polymer. In other embodiments, substantially all of the recurring units of Formula (I) can be oriented at or near the interior of the polymer and substantially all of the recurring units of Formula (II) can be oriented at or near the exterior of the polymer. In some embodiments, about 50% to about 98%, about 60% to about 95%, about 70% to about 95%, about 80% to about 90%, >about 80% or >about 90% of the recurring units of Formula (I) can be oriented at or near the interior of the polymer. In some embodiments, about 50% to about 98%, about 60% to about 95%, about 70% to about 95%, about 80% to about 90%, >about 80% or >about 90% of the recurring units of Formula (II) can be oriented at or near the exterior of the polymer.

In some embodiments, one or more recurring units of Formula (VII) may be oriented at or near the interior of the polymer. In other embodiments, one or more recurring units of Formula (VII) may be oriented at or near the exterior of the polymer. In some embodiments, one or more recurring units of Formula (I) can be oriented at or near the interior of the polymer, one or more recurring units of Formula (II) can be oriented at or near the exterior of the polymer, and one or more recurring units of Formula (VII) can be oriented at or near the exterior of the polymer. In other embodiments, substantially all of the recurring units of Formula (I) can be oriented at or near the interior of the polymer, substantially all of the recurring units of Formula (II) can be oriented at or near the exterior of the polymer, and substantially all of the recurring units of Formula (VII) can be oriented at or near the exterior of the polymer. In some embodiments, about 50% to about 98%, about 60% to about 95%, about 70% to about 95%, about 80% to about 90%, >about 80% or >about 90% of the recurring units of Formula (I) can be oriented at or near the interior of the polymer. In some embodiments, about 50% to about 98%, about 60% to about 95%, about 70% to about 95%, about 80% to about 90%, >about 80% or >about 90% of the recurring units of Formula (II) can be oriented at or near the exterior of the polymer. In some embodiments, about 50% to about 98%, about 60% to about 95%, about 70% to about 95%, about 80% to about 90%, >about 80% or >about 90% of the recurring units of Formula (VII) can be oriented at or near the exterior of the polymer.

In some embodiments, the nucleic acid may be associated with a side chain moiety of the polymer. In other embodiments, the nucleic acid may be associated with an end or terminal recurring unit of the polymer. In yet other embodiments, the nucleic acid may be associated with the middle of the polymer. In still yet other embodiments, the nucleic acid may be associated with the backbone of the polymer. In some embodiments, the nucleic acid may be associated with an exterior moiety or moieties or an exterior surface of the polymer. In some embodiments, the nucleic acid may be associated with an interior moiety or moieties or an interior surface of the polymer. In some embodiments, the nucleic acid can be at least partially contained within the polymer. In other embodiments, the nucleic acid may be substantially completely contained within the polymer. In some embodiments, one or more recurring units of Formula (I) can be oriented at or near the interior of the polymer, one or more recurring units of Formulae (II) and/or (VII) can be oriented at or near the exterior of the polymer, and the nucleic acid can be at least partially contained within the polymer. In some embodiments, substantially all of the recurring units of Formula (I) can be oriented at or near the interior of the polymer, substantially all of the recurring units of Formulae (II) and/or (VII) can be oriented at or near the exterior of the polymer, and the nucleic acid can be at least partially contained within the polymer. In some embodiments, about 50% to about 98%, about 60% to about 95%, about 70% to about 95%, about 80% to about 90%, >about 80% or >about 90% of the recurring units of Formula (I) can be oriented at or near the interior of the polymer, and the nucleic acid can be at least partially contained within the polymer. In some embodiments, about 50% to about 98%, about 60% to about 95%, about 70% to about 95%, about 80% to about 90%, >about 80% or >about 90% of the recurring units of Formula (II) can be oriented at or near the exterior of the polymer, and the nucleic acid can be at least partially contained within the polymer. In some embodiments, about 50% to about 98%, about 60% to about 95%, about 70% to about 95%, about 80% to about 90%, >about 80% or >about 90% of the recurring units of Formula (VII) can be oriented at or near the exterior of the polymer, and the nucleic acid can be at least partially contained within the polymer. Advantageously, in some embodiments, the recurring unit(s) of Formula (II) can help the polymer disrupt the endosomal membrane to penetrate the cell wall, while the recurring unit(s) of Formula (I) can associate with the nucleic acid and/or shield it from the endosomal membrane. Advantageously, in some embodiments, the recurring unit(s) of Formula (VII) can cause the polymer to become relatively insoluble and/or hydrophobic at a location having a pH above and/or below the transition point, such as at or near a tumor. Those skilled in the art will recognize that the location and orientation of association may otherwise vary depending on the properties of the specific recurring units and nucleic acid.

Polymers disclosed herein can be prepared in various ways. In some embodiments, a first reactant that can be a homopolymer including a first recurring unit of Formula (I) or salt thereof can be intermixed with a second reactant that includes R¹. In some embodiments, the first reactant can be poly-L-lysine (PLL). The molecular weight of the PLL can vary widely. In some embodiments, the molecular weight of the PLL can be in the range of from about 10 kDa to about 30 kDa. In other embodiments, the molecular weight of the PLL can be in the range of from about 10 kDa to about 20 kDa. In yet other embodiments, the molecular weight of the PLL can be in the range of from about 20 kDa to about 30 kDa. In other embodiments, the molecular weight of the PLL can be in the range of from about 15 kDa to about 25 kDa. In some embodiments, the first reactant can include at least 95 mole % of recurring units of Formula (I) based on the total number of recurring units in the first reactant. In some embodiments, the second reactant can be an optionally substituted C₄-C₂₄ fatty acid (e.g., nonanoic acid, oleic acid, lauric acid, myristoleic acid, palmitoleic acid, margaric acid, stearic acid, arachidic acid, behenic acid, or lignoceric acid) or an optionally substituted sterol. In some embodiments, the second reactant can be oleic acid. In other embodiments, the second reactant can be nonanoic acid. In some embodiments, the first reactant and the second reactant are reacted together via a coupling reaction.

In some embodiments, the first reactant can be intermixed with about 0.01 to about 0.7 equivalents of the second reactant, so that about 1% to about 70% of the recurring units of the resulting polymer can be the second recurring unit of Formula (II). In other embodiments, the first reactant can be intermixed with about 0.01 to about 0.2 equivalents of the second reactant, so that about 1% to about 20% of the recurring units of the resulting polymer can be the second recurring unit of Formula (II).

In yet other embodiments, the first reactant can be a monomer that forms a recurring unit of Formula (I) and the second reactant can be a monomer that forms a recurring unit of Formula (II). The monomers can be polymerized to form a copolymer using methods known to those skilled in the art. In some embodiments, a monomeric first reactant can be intermixed with about 0.01 to about 0.7 equivalents of a monomeric second reactant. In other embodiments, a monomeric first reactant can be intermixed with about 0.01 to about 0.2 equivalents of a monomeric second reactant.

Polymers that can include a first recurring unit of Formula (I), a second recurring unit of Formula (II), and a third recurring unit of Formula (VII) as disclosed herein can be prepared in various ways. In some embodiments, a first reactant that can be a homopolymer including a first recurring unit of Formula (I) or salt thereof can be intermixed with a second reactant that includes R¹ and a third reactant that includes R⁴. In some embodiments, the first reactant can be poly-L-lysine. The molecular weight of the PLL can vary widely. In some embodiments, the molecular weight of the PLL can be in the range of from about 10 kDa to about 30 kDa. In other embodiments, the molecular weight of the PLL can be in the range of from about 10 kDa to about 20 kDa. In yet other embodiments, the molecular weight of the PLL can be in the range of from about 20 kDa to about 30 kDa. In other embodiments, the molecular weight of the PLL can be in the range of from about 15 kDa to about 25 kDa. In some embodiments, the first reactant can include at least 95 mole % of recurring units of Formula (I) based on the total number of recurring units in the first reactant. In some embodiments, the second reactant can be an optionally substituted C₄-C₂₄ fatty acid (e.g., nonanoic acid, oleic acid, lauric acid, myristoleic acid, palmitoleic acid, margaric acid, stearic acid, arachidic acid, behenic acid, or lignoceric acid) or an optionally substituted sterol. In some embodiments, the second reactant can be oleic acid. In other embodiments, the second reactant can be nonanoic acid. In yet other embodiments, the third reactant can include a pH transition group and a group that is reactive with the first reactant. For example, the third reactant can include a succinate moiety. In another example, the third reactant can be succinic acid or succinic anhydride. In yet another example, the third reactant can include an amine moiety, such as those described herein. In some embodiments, the third reactant can be morpholine moiety, for example, 4-(2-isothiocyanatoethyl)morpholine. In some embodiments, one or more of the reactants (for example, the first second and third reactants) are reacted together via a coupling reaction.

In some embodiments, the first reactant can be intermixed with about 0.01 to about 0.7 equivalents of the second reactant, so that about 1% to about 70% of the recurring units of the resulting polymer can be the second recurring unit of Formula (II); and with about 0.01 to about 0.9 equivalents of the third reactant, so that about 1% to about 90% of the recurring units of the resulting polymer can be the third recurring unit of Formula (VII). In other embodiments, the first reactant can be intermixed with about 0.01 to about 0.2 equivalents of the second reactant, so that about 1% to about 20% of the recurring units of the resulting polymer can be the second recurring unit of Formula (II); and with about 0.25 to about 0.85 equivalents of the third reactant, so that about 25% to about 85% of the recurring units of the resulting polymer can be the third recurring unit of Formula (VII).

In other embodiments, the first reactant can be a monomer that forms a recurring unit of Formula (I), the second reactant can be a monomer that forms a recurring unit of Formula (II), and the third reactant can be a monomer that forms a recurring unit of Formula (VII). The monomers can be polymerized to form a copolymer using methods known to those skilled in the art. In some embodiments, a monomeric first reactant can be intermixed with about 0.1 to about 0.7 equivalents of a monomeric second reactant and about 0.1 to about 0.7 equivalents of a monomeric third reactant. In other embodiments, a monomeric first reactant can be intermixed with about 0.1 to about 0.2 equivalents of a monomeric second reactant and about 0.1 to about 0.4 equivalents of a monomeric third reactant.

Those skilled in the art appreciate that the first, second, and third reactants can be intermixed in any order. In some embodiments, the first reactant can be mixed with the second reactant, and then the third reactant can be added. In some embodiments, the first reactant, the second reactant, and the third reactant can be intermixed at substantially the same time. In some embodiments, the first reactant can be intermixed with about 0.01 to about 0.7 equivalents of the second reactant, so that about 1% to about 70% of the recurring units of the resulting polymer can be the second unit of Formula (II). In other embodiments, the first reactant can be intermixed with about 0.01 to about 0.7 equivalents of the third reactant, so that about 1% to about 70% of the recurring units of the resulting polymer can be the third recurring unit of Formula (VII). In yet other embodiments, the first reactant can be intermixed with about 0.01 to about 0.2 equivalents of the second reactant, so that about 1% to about 20% of the recurring units of the resulting polymer can be the second unit of Formula (II). In other embodiments, the first reactant can be intermixed with about 0.01 to about 0.4 equivalents of the third reactant, so that about 1% to about 40% of the recurring units of the resulting polymer can be the third recurring unit of Formula (VII).

In some embodiments, the reactants can be intermixed with one or more solvents, such as an organic solvent. Examples of organic solvents include, but are not limited to, dimethylformamide (DMF) and dimethyl sulfoxide (DMSO). The reactants and/or solvents may be commercially available and/or may be synthesized according to methods known to those of ordinary skill in the art as guided by the teachings provided herein.

In some embodiments, the reactants can be intermixed in the presence of a suitable base. Suitable bases are known to those skilled in the art. Examples of bases include, but are not limited to, an amine base, such as an alkylamine (including mono-, di- and tri-alkylamines (e.g., triethylamine)).

In some embodiments, the reactants can be intermixed in the presence of a coupling agent. Any suitable coupling agent may be used. In some embodiments, the coupling agent can be selected from 1-ethyl-3-(3-dimethylaminopropyl)-carbodiimide (EDC), 1,3-dicyclohexyl carbodiimide (DCC), 1,1′-carbonyl-diimidazole (CDI), N,N′-disuccinimidylcarbonate (DSC), N-[(dimethylamino)-1H-1,2,3-triazolo-[4,5-b]pyridine-1-yl-methylene]-N-methylmethanaminium hexafluorophosphate N-oxide (HATU), 2-[(1H-benzotriazol-1-yl)-1,1,3,3-tetramethylaminium hexafluorophosphate (HBTU), 2-[(6-chloro-1H-benzotriazol-1-yl)-1,1,3,3-tetramethylaminium hexafluorophosphate (HCTU), benzotriazole-1-yl-oxy-tris-pyrrolidino-phosphonium hexafluorophosphate (PyBOP®), bromo-tris-pyrrolidino-phosphonium hexafluorophosphate (PyBroP®), 2-[(1H-benzotriazol-1-yl)-1,1,3,3-tetramethylaminium tetrafluoroborate (TBTU), and benzotriazol-1-yl-oxy-tris-(dimethylamino)phosphonium hexafluorophosphate (BOP).

In some embodiments, the reaction may be carried out at room temperature. In some embodiments, the reaction mixture may be stirred for several hours. The reaction products may be isolated by any means known in the art including chromatographic techniques. In some embodiments, the solvent may be evaporated to recover the reaction product (e.g., via rotary evaporation). In other embodiments, the reaction product may be removed by precipitation followed by centrifugation.

A wide variety of polymers comprising the recurring units described herein may be made by varying the molecular weight and structure of the first reactant, the molecular weight and structure of the second reactant, the molecular weight and structure of the third reactant, the size and type of the R¹ groups on the second reactant, the size and type of the R⁴ groups on the third reactant, the mole ratios of the first reactant to second reactant, and/or the mole ratios of first reactant to third reactant. In addition, mixtures of different first reactants and/or mixtures of different second reactants and/or mixtures of different third reactants may be used.

Furthermore, additional reactants may advantageously be used to incorporate additional recurring units into the polymer. For example, additional reactants (e.g., capable of reacting with the NH₂ group of a recurring unit of Formula (I) and/or capable of polymerizing with a reactant (e.g., the first reactant)) can be intermixed with the reaction mixture.

The polymers described herein that are associated with a nucleic acid (e.g., siRNA) can be administered to a human patient per se, or in pharmaceutical compositions where they are mixed with one or more suitable pharmaceutical carriers and/or excipient(s). Some embodiments herein provide a pharmaceutical composition that can include one or more polymers described herein associated with a nucleic acid, and further include at least one selected from a pharmaceutically acceptable excipient, a pharmaceutical carrier, and a diluent.

The term “pharmaceutical composition” refers to a mixture of a polymer associated with a nucleic acid disclosed herein with one or more other chemical components, such as diluents or additional pharmaceutical carriers. The pharmaceutical composition facilitates administration of the polymer and/or the nucleic acid to an organism.

Multiple techniques of administering a pharmaceutical composition exist in the art. Suitable routes of administration may include, for example, parenteral delivery, including intramuscular, subcutaneous, intravenous, intramedullary injections, as well as intrathecal, direct intraventricular, intraperitoneal, intranasal, or intraocular injections. The polymer associated with a nucleic acid can also be administered in sustained or controlled release dosage forms, including depot injections, osmotic pumps, and the like, for prolonged and/or timed, pulsed administration at a predetermined rate. Additionally, the route of administration may be local or systemic.

The term “pharmaceutical carrier” refers to a chemical compound that facilitates the incorporation of a polymer associated with a nucleic acid into cells or tissues.

The term “diluent” refers to chemical compounds diluted in water that will dissolve the polymer with an associated nucleic acid as well as stabilize the biologically active form of the polymer with the associated nucleic acid. Salts dissolved in buffered solutions are utilized as diluents in the art. As used herein, an “excipient” refers to an inert substance that is added to a polymer with an associated nucleic acid to provide, without limitation, bulk, consistency, stability, binding ability, lubrication, disintegrating ability, etc., to the composition. A “diluent” is a type of excipient.

The term “physiologically acceptable” refers to a pharmaceutical carrier or diluent that does not abrogate the biological activity and properties of the polymer or the nucleic acid.

Techniques for formulation and administration of the pharmaceutical compositions of the instant application may be found in “Remington's Pharmaceutical Sciences,” Mack Publishing Co., Easton, Pa., 18th edition, 1990. The pharmaceutical compositions may be manufactured in a manner that is itself known. Pharmaceutical compositions may be formulated in any conventional manner using one or more physiologically acceptable pharmaceutical carriers comprising excipients and auxiliaries which facilitate processing of the active compounds into preparations which can be used pharmaceutically. Proper formulation is dependent upon the route of administration chosen. Any of the well-known techniques, pharmaceutical carriers, and excipients may be used as suitable and as understood in the art; e.g., in Remington's Pharmaceutical Sciences, above.

Some embodiments herein are directed to a method of delivering a nucleic acid, such as siRNA, to a cell. In some embodiments, the polymers described herein can be used to transfect a cell. These embodiments can include delivering the nucleic acid via the polymer to the cell, for example by contacting the cell with the polymer. Suitable cells for use according to the methods described herein include prokaryotes, yeast, or higher eukaryotic cells, including plant and animal cells (e.g., mammalian cells). In some embodiments, the cells can be tumor cells, such as animal (e.g., mammalian and/or human) tumor cells. Cells lines which are model systems for tumors may be used. In some embodiments these methods can be performed in vitro, while in other embodiments they can be performed in vivo.

Other embodiments are directed to a method of treating a mammal. These embodiments may include identifying a mammal in need of gene therapy and administering to the mammal a polymer associated with a nucleic acid as described herein.

Some embodiments disclosed herein are directed to a method for treating a tumor that can include administering an effective amount of a polymer associated with a nucleic acid as described herein. Other embodiments disclosed herein are directed to a method for treating a tumor that can include contacting a tumor cell with an effective amount of a polymer associated with a nucleic acid as described herein. Treatment of a tumor can include shrinking the tumor and/or killing or damaging some or all of the tumor cells. The tumor can be benign, pre-malignant, or malignant (e.g., cancerous). The tumor can be solid or non-solid (e.g., dispersed). Examples of solid tumors include, but are not limited to, those associated with lung cancer, breast cancer, ovarian cancer, prostate cancer, colorectal cancer, brain cancer, testicular cancer, pancreatic cancer, liver cancer, and stomach cancer. Other examples of solid tumors include, but are not limited to, sarcomas, carcinomas, melanomas, and lymphomas. In some embodiments, the nucleic acid can treat the tumor.

The pharmaceutical compositions described herein may be administered to the subject by any suitable means. Non-limiting examples of methods of administration include, among others, (a) administration via injection, subcutaneously, intraperitoneally, intravenously, intramuscularly, intradermally; intraorbitally, intracapsularly, intraspinally, intrasternally, or the like, including infusion pump delivery; (b) administration locally such as by injection directly in the renal or cardiac area, e.g., by depot implantation; as well as deemed appropriate by those of skill in the art for bringing the polymer associated with a nucleic acid into contact with living tissue.

Pharmaceutical compositions suitable for administration include polymers where the active ingredients (e.g., associated nucleic acid) are contained in an amount effective to achieve its intended purpose. The effective amount of the nucleic acids and polymers disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. More specifically, a therapeutically effective amount means an amount of nucleic acid effective to prevent, alleviate or ameliorate symptoms of disease or prolong the survival of the subject being treated. Determination of a therapeutically effective amount is well within the capability of those skilled in the art, especially in light of the detailed disclosure provided herein.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration.

Polymers disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. When selecting a model to determine efficacy, the skilled artisan can be guided by the state of the art to choose an appropriate model, dose, and route of administration, and regime.

### EXAMPLES

Additional embodiments are disclosed in further detail in the following examples, which are not in any way intended to limit the scope of the claims. All chemicals and other reagents were purchased from Sigma-Aldrich.

### Example 1

### Polymer Synthesis

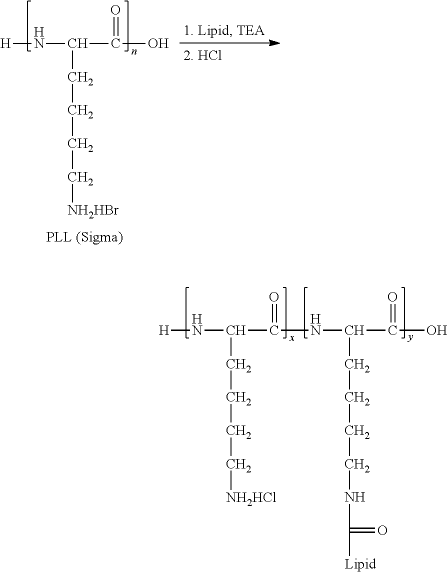

TABLE 1
PLL polymers with lipid

| POLYMER | POLY-L-LYSINE (PLL) | LIPID |
|---|---|---|
| PLL 1 | 99 mole % (25 kDa) | 1 mole % (oleyl) |
| PLL 2 | 95 mole % (25 kDa) | 5 mole % (oleyl) |
| PLL 3 | 90 mole % (12 kDa) | 10 mole % (oleyl) |
| PLL 4 | 80 mole % (12 kDa) | 20 mole % (oleyl) |
| PLL 5 | 90 mole % (12 kDa) | 10 mole % (cholesterol) |
| PLL 6 | 80 mole % (12 kDa) | 20 mole % (cholesterol) |
| PLL 7 | 90 mole % (25 kDa) | 10 mole % (oleyl) |
| PLL 8 | 80 mole % (25 kDa) | 20 mole % (oleyl) |
| PLL 9 | 90 mole % (25 kDa) | 10 mole % (cholesterol) |
| PLL 10 | 80 mole % (25 kDa) | 20 mole % (cholesterol) |

### Polymer PLL 1:

Poly-L-lysine having a molecular weight of 25 kDa (PLL-25 kDa, 100 mg) was partially dissolved in dimethylformamide (DMF) (3 mL). Triethylamine (TEA, 1 mL) was added. The reaction mixture was sonicated for 2 minutes. Oleyl-NHS (1.8 mg) was added into the reaction mixture. The reaction was stirred for 18 hours and quenched with water (5 mL). The reaction was stirred for another 2 hours and acidified with diluted HCl solution until about pH 2. The reaction mixture was dialyzed against water for 1 day, and the water was changed (4 L×4 times). The product was lyophilized. Polymer PLL 1 was obtained (58 mg, 72% yield) and characterized by a ninhydrin test and ¹H-NMR.

### Polymer PLL 2:

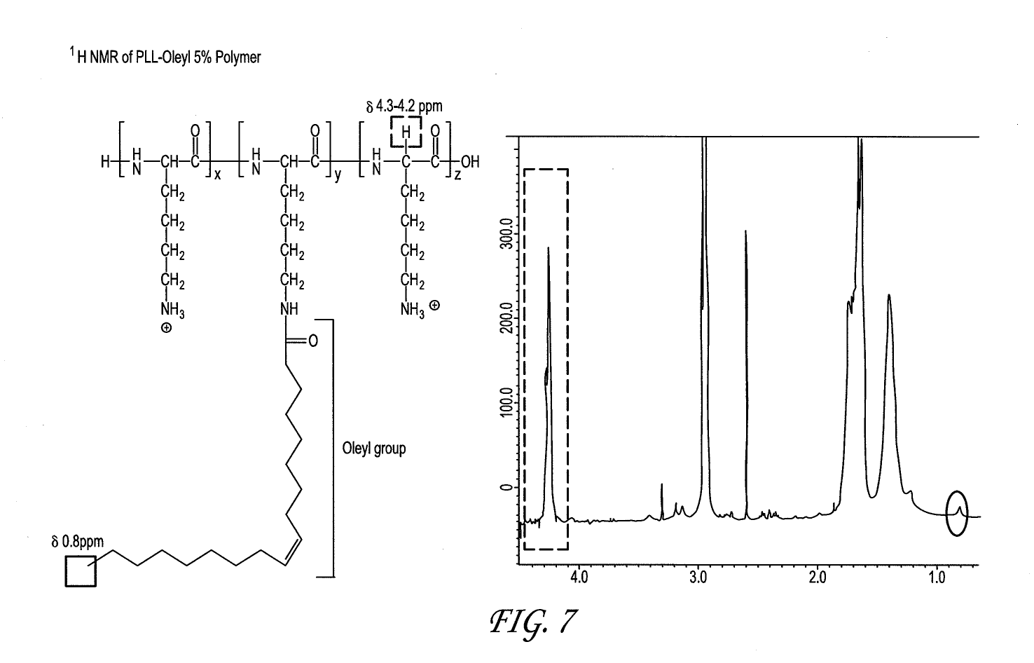

Polymer PLL 2 was prepared in a similar manner as PLL 1, using 9.1 mg of oleyl-NHS. Polymer PLL 2 was obtained (72 mg, 85% yield) and characterized by a ninhydrin test and ¹H-NMR. Representative peaks indicating the formation of polymer PLL 2 include the following (in D₂O solvent): δ 0.8 ppm (lipid —CH₃); δ 4.3-4.2 ppm (amine C—H). See FIG. 7.

### Polymers PLL 3-PLL 10:

Polymers PLL 3-PLL 10 were prepared and characterized in a similar manner as Polymers PLL 1 and PLL 2, but with the respective starting materials and amounts specified in Table 1.

### Example 2

### Synthesis of Polymers Having pH Transition Point

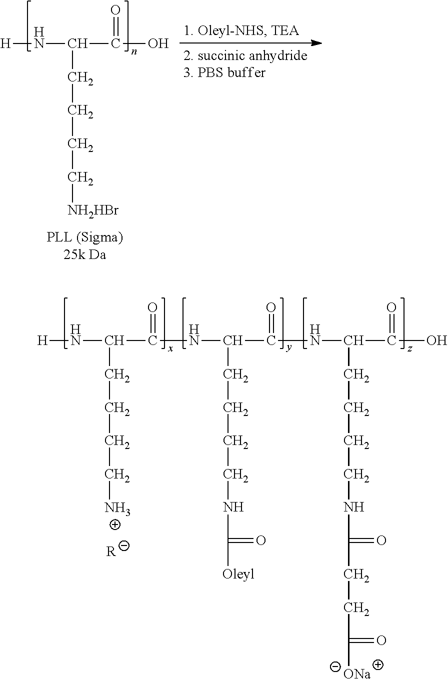

TABLE 2
PLL polymers with lipid and succinic acid

| | 25 kDa | LIPID (OLEYL) | SUCCINIC |
|---|---|---|---|
| POLYMER | PLL UNIT | UNIT | ACID UNIT |
| PLL/PTP 1 | 45 mole % | 5% mole % | 50 mole % |
| PLL/PTP 2 | 40 mole % | 10 mole % | 50 mole % |
| PLL/PTP 3 | 25 mole % | 5% mole % | 70 mole % |
| PLL/PTP 4 | 20 mole % | 10% mole % | 70 mole % |

Polymer PLL/PTP 1:

PLL-25 kDa (25 mg) was partially dissolved in DMF (3 mL). Triethylamine (TEA, 300 μL) was added. The reaction mixture was sonicated for 2 minutes. Oleyl-NHS (2.3 mg) and succinic anhydride (6.0 mg) were added into the reaction mixture. The reaction was stirred for 18 hours and quenched with PBS buffer (pH 5.5, 0.2 M, 10 mL). The reaction was stirred for another 2 hours and acidified with diluted HCl solution until about pH 7. The reaction mixture was dialyzed against water for 1 day and the water was changed (4 L×4 times). The reaction mixture was further acidified to pH 2 with diluted HCl solution. The mixture was dialyzed against water for 1 more day, and the water was changed (4 L×4 times). The product was lyophilized. Polymer PLL/PTP1 was obtained (14 mg, 58% yield) and characterized by a ninhydrin test and ¹H-NMR.

### Polymer PLL/PTP 2:

Polymer PLL/PTP 2 was prepared in a similar manner as PLL/PTP 1, with 4.5 mg of oleyl-NHS. Polymer PLL/PTP 2 was obtained (18 mg, 72% yield) and characterized by a ninhydrin test and ¹H-NMR.

### Polymer PLL/PTP 3:

Polymer PLL/PTP 3 was prepared in a similar manner as PLL/PTP 1, with 2.3 mg of oleyl-NHS and 8.4 mg succinic anhydride. Polymer PLL/PTP 3 was obtained (12 mg, 44% yield) and characterized by a ninhydrin test and ¹H-NMR.

### Polymer PLL/PTP 4:

Polymer PLL/PTP 4 was prepared in a similar manner as PLL/PTP 1, with 4.5 mg oleyl-NHS and 8.4 mg succinic anhydride. Polymer PLL/PTP 4 was obtained (14 mg, 49% yield) and characterized by ninhydrin test and ¹H-NMR.

### Example 3

### Synthesis of Polymers Having pH Transition Point

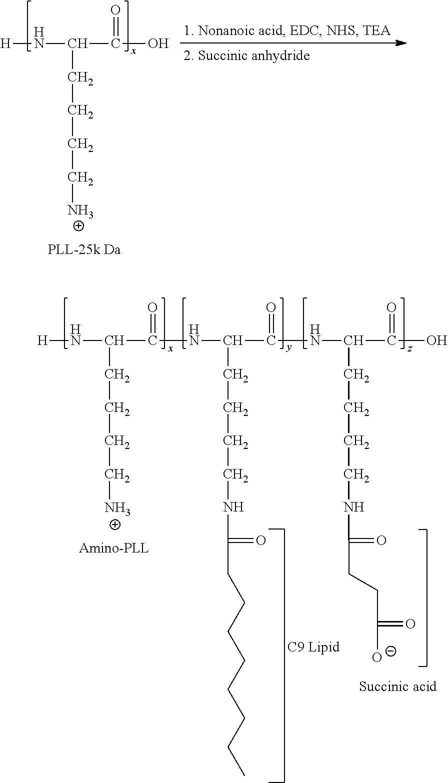

TABLE 3
PLL polymers with lipid and succinic acid

| | LIPID (C₉) | SUCCINIC | 25 kDa |
|---|---|---|---|
| POLYMER | UNIT | ACID UNIT | PLL UNIT |
| PLL/PTP 5 | 5 mole % | 10 mole % | 85 mole % |
| PLL/PTP 6 | 5 mole % | 25 mole % | 70 mole % |
| PLL/PTP 7 | 5 mole % | 50 mole % | 45 mole % |
| PLL/PTP 8 | 5 mole % | 85 mole % | 10 mole % |

Polymer PLL/PTP 5:

PLL-25 kDa (50 mg) was partially dissolved in DMF (4 mL). Triethylamine (TEA, 50 μL) was added. The reaction mixture was sonicated for 2 minutes. A solution of nonanoic acid (2 mg), 1-ethyl-3-(3-dimethylaminopropyl)carbodiimide) (EDC, 3.2 mg), and N-hydroxysuccinimide (NHS, 3.0 mg) in DMF (1 mL) was added into the solution. The reaction mixture was stirred for 18 hours. Succinic anhydride (2.4 mg) was added to the mixture, and the mixture was stirred for another 18 hours. The reaction was quenched with water (5 mL) and stirred for 30 minutes. The reaction mixture was acidified with a diluted hydrochloric acid solution until about pH 3. The reaction mixture was dialyzed against water overnight and the water was changed (4 L×4 times). Polymer PLL/PTP 5 was obtained after lyophilization (31 mg) and characterized by a ninhydrin test and ¹H-NMR.

### Polymer PLL/PTP 6:

Polymer PLL/PTP 6 was prepared in a similar manner as polymer PLL/PTP 5, with 6.0 mg succinic anhydride. Polymer PLL/PTP 6 was obtained after lyophilization (38 mg) and characterized by a ninhydrin test and ¹H-NMR.

### Polymer PLL/PTP 7:

Polymer PLL/PTP 7 was prepared in a similar manner as polymer PLL/PTP 5, with 12.0 mg succinic anhydride. Polymer PLL/PTP 7 was obtained after lyophilization (41 mg) and characterized by a ninhydrin test and ¹H-NMR.

### Polymer PLL/PTP 8:

Polymer PLL/PTP 8 was prepared in a similar manner as polymer PLL/PTP 5, with 20.3 mg succinic anhydride. Polymer PLL/PTP 8 was obtained after lyophilization (62 mg) and characterized by a ninhydrin test and ¹H-NMR.

### Example 4

### Association of Polymers with siRNA

Polymers PLL 1-10:

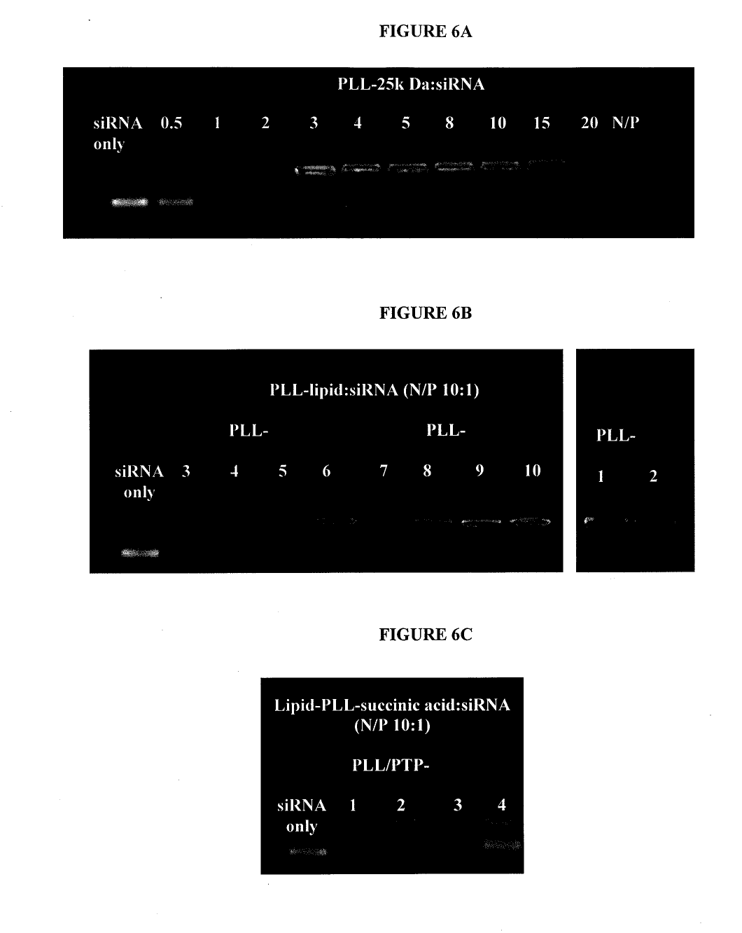

Several samples of a 25 kDa PLL homopolymer were intermixed with the double-stranded siRNA described in SEQ ID NO:1 and SEQ ID NO:2 at various N/P ratios, as indicated in FIG. 6A. The polymer/siRNA mixtures were subjected to a gel retardation assay.

The results of the gel retardation assay, illustrated in FIG. 6A, indicate that siRNA migrates more slowly as the relative amount of polymer increases. These results indicate that the PLL homopolymer was associated with siRNA.

Polymers PLL 1-10 were each intermixed with the double-stranded siRNA described in SEQ ID NO:1 and SEQ ID NO:2 at an N/P ratio of 10:1, as indicated in FIG. 6B. The polymer/siRNA mixtures were subjected to a gel retardation assay.

The results of the gel retardation assay, illustrated in FIG. 6B, indicate that the siRNA intermixed with a polymer migrated more slowly than the control siRNA sample, which had not been intermixed with a polymer. These results indicate that the Polymers PLL 1-10 were associated with siRNA.

### Polymers PLL/PTP 1-4:

Polymers PLL/PTP 1-4 were each intermixed with the double-stranded siRNA described in SEQ ID NO:1 and SEQ ID NO:2 at an N/P ratio of 10:1. The polymer/siRNA mixtures were subjected to a gel retardation assay.

The results of the gel retardation assay, illustrated in FIG. 6C, indicated that the siRNA intermixed with a polymer migrated more slowly than the control siRNA sample, which had not been intermixed with a polymer. In addition, the mixture that included PLL/PTP 4, showed some signs of the presence of free siRNA, as evidenced by the bright band near the bottom of the lane. The results with PLL/PTP 4 indicate that the succinic acid may compete with the siRNA to associate with the polymer, and at higher concentrations can displace the siRNA (leading to an increased amount of free siRNA at higher concentrations of succinic acid). These results indicate that the Polymers PLL/PTP 1-4 were associated with siRNA.

### Example 5

### siRNA Transfection

Cells (HT1080 cells or CHO-K1 cells) were seeded in 96-well plates at a density of 1×10⁴ cells per well one day before the transfection. siRNA (1.0 μg) was dissolved in distilled water and further diluted to 30 μL with OptiMEM (Invitrogen) to form a solution. Solutions of selected polymers were prepared by dissolving the selected polymers in dH₂O to a concentration of 5 mg/mL. The diluted siRNA solution and the polymer solutions were mixed and incubated at room temperature for 15 min. The mixture of the siRNA and the polymer (15 μL) were added to each well of the pre-seeded cells, mixed, and incubated at 37° C. incubator with 5% CO₂.

After about 48 hours, transfection was evaluated by measuring the expression of GFP under a fluorescence microscope. The absorbance of GFP was detected at 485-528 nm using a UV-vis microplate reader.

The siRNA was a double stranded siRNA sequence with 21-mer targeting dscGFP. The sequences are as follows:

(SEQ ID NO: 1)
Sense: 5′-GUGAUCUUCACCGACAAGATT
(SEQ ID NO: 2)
Anti-sense: 5′-UCUUGUCGGUGAAGAUCACTT

### Example 6

### Plasmid DNA (Gene) Transfection

### Cell Culture:

CHO-K1 cells were maintained in F-12 medium containing 10% FBS, 100 units/mL penicillin and 100 μg/mL streptomycin at 37° C., 5% CO₂ and 100% humidity conditions. The cells were split every 3-4 days to avoid confluency.

### Plasmid DNA Preparation:

pEGFP-N1 plasmid DNA (GenBank Accession #U55762; SEQ. ID. NO.: 3) was purchased from BD Sciences Clontech company, which encodes a red-shifted variant of wild-type GFP that has been optimized for brighter fluorescence and higher expression in mammalian cells. The GFP protein was controlled by an immediate early promoter of CMV (P_{CMV IE}). The plasmids were amplified in DH5 E. coli and purified with Qiagen Plasmid Max Preparation Kit, and had an A260/A280 greater than 1.7.

### In Vitro Transfection:

CHO-K1 cells were plated in 96-well tissue culture plates (1×10⁵ cells/well for CHO-K1 cells) and incubated overnight in F-12K medium with 10% FBS. For each well, an aliquot of 7.5 μL polymer solution in Opti-MEM at different concentrations was added to 7.5 μL DNA solution containing 0.15 μg of pEGFP-N1 plasmid in Opti-MEM and mixed. The DNA and polymer mixture were incubated for 15 minutes at room temperature to allow for the formation of DNA-polymer complexes. The complexes were added to each well and incubated at 37° C., 5% CO₂ for 48 hours. The EGFP gene transfection efficiency was determined by GFP signal analysis. CarriGene (Kinovate Life Science) was used as a positive control according to the protocol provided by manufacturer.

### Observation of GFP Signal:

Green fluorescent signal in transfected cells was observed under a fluorescent microscope (Olympus, filter 520 nm). The cells were photographed using a 10× objective.

In order to quantify the transfection efficiency, the relative fluorescent unit of transfected cells was determined by fluorescent microplate reader (FLX 800, Bio-TEK Instruments Co Ltd).

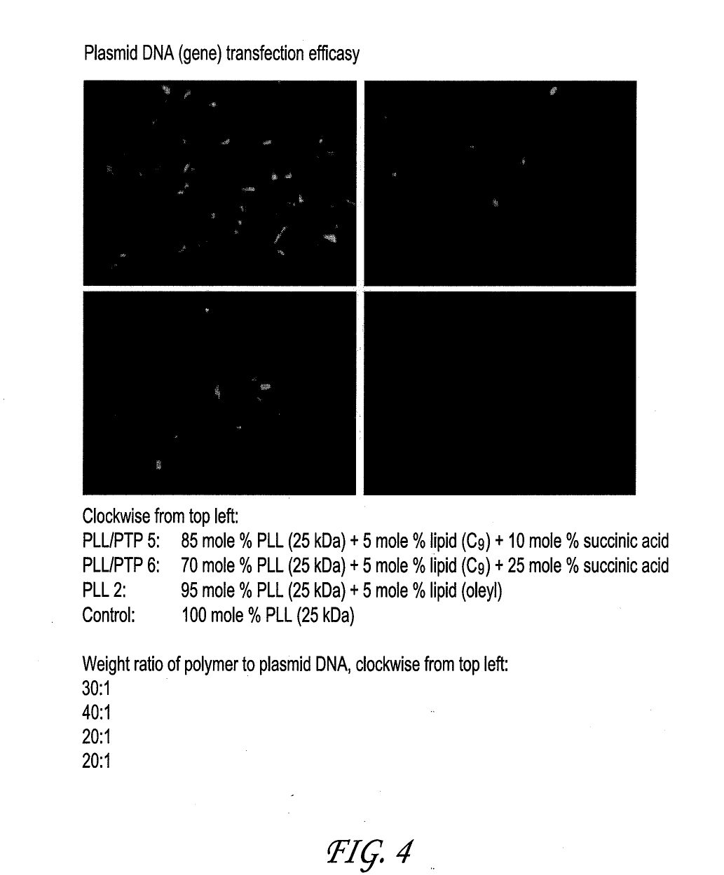

Polymers tested included: PLL/PTP 5, PLL/PTP 6, PLL 2, and a control polymer (PLL). The results are shown in FIG. 4. FIG. 4 indicates that polymer PLL/PTP 5 exhibited relatively high GFP expression, and accordingly, relatively high transfection efficiency, as compared to the control polymer.

### Example 7

### Cell Viability Assay

A solution of 3-(4,5-dimethylthiazol-2-yl)-2,5-diphenyltetrazolium bromide (MTT) was prepared by dissolving 250 mg of solid MTT in 50 mL of Dubecco PBS. The solution was stored at 4° C. After 48 hours of transfection as described in Example 6, MTT solution (10 μl, of the 5 mg/mL) was added to each well of the cells and incubated at 37° C. for 2-4 hours until purple crystal growth could be observed. The solubilized solution (100 μL) was added and incubated at 37° C. overnight. The absorbance was detected at wavelength of 570 nm with the absorbance at 690 nm used as reference.

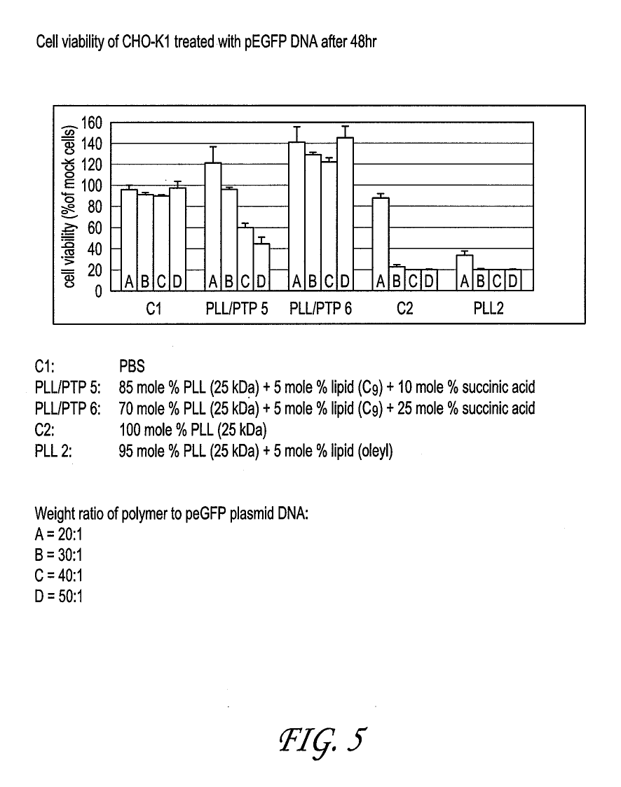

The results of the cell viability assay for polymers C1, PLL/PTP 5, PLL/PTP 6, C2, and PLL 2 at weight ratios of polymer to DNA of 20:1, 30:1, 40:1, and 50:1 are shown in FIG. 5. As illustrated in FIG. 5, polymer PLL/PTP 6 at all four weight ratios exhibited greater cell viability than the control compounds C1 and C2.

It will be understood by those of skill in the art that numerous and various modifications can be made without departing from the spirit of the present application. Therefore, it should be clearly understood that the forms of the present invention are illustrative only and not intended to limit the scope of the present invention.

What is claimed is:

1. A polymer comprising
a first recurring unit of Formula (I) having the structure: 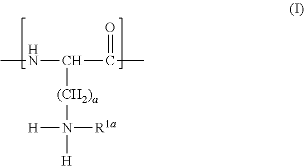
a second recurring unit of Formula (II) having the structure: 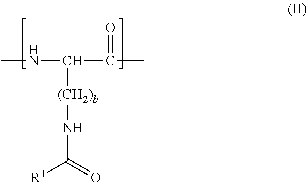
wherein
each a and each b are independently 1, 2, 3, 4, 5 or 6;
R^{1a} is absent or H, and if R^{1a} is H, the nitrogen atom to which R^{1a} is attached has an associated positive charge; and
R¹ is selected from the group consisting of C₄-C₂₄ alkyl, C₄-C₂₄ alkenyl and an optionally substituted steryl.

2. The polymer of claim 1, wherein a is 4.

3. The polymer of claim 1, wherein R^{1a} is H.

4. The polymer of claim 1, wherein R¹ is C₄-C₂₄ alkyl.

5. The polymer of claim 1, wherein R¹ is selected from the group consisting of oleyl, lauryl, myristyl, palmityl, margaryl, stearyl, arachidyl, behenyl, lignoceryl and an optionally substituted steryl.

6. The polymer of claim 1, wherein b is 4 and R¹ is —(CH₂)₇CH═CH(CH₂)₇CH₃.

7. The polymer of claim 1, wherein b is 4 and R¹ is C₈ alkyl.

8. The polymer of claim 1, further comprising a third recurring unit of Formula (VII) having the structure: 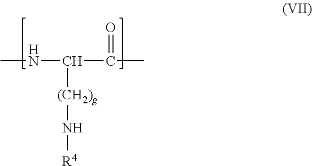
wherein
each g is independently 1, 2, 3, 4, 5 or 6; and
R⁴ comprises a group that has a pH transition point.

9. The polymer of claim 8, wherein g is 4.

10. The polymer of claim 8, wherein R⁴ is hydrophilic at a pH≧the transition point.

11. The polymer of claim 8, wherein R⁴ is hydrophobic at a pH<the transition point.

12. The polymer of claim 8, wherein R⁴ is a group that comprises. an imidazolyl group, a 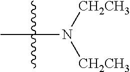
group, a 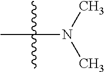
group or a morpholinyl group.

13. The polymer of claim 8, wherein R⁴ has the following structure: —C(═O)—(CH₂)₁₋₄—C(═O)OR^{A} at a pH≧the transition point, wherein R^{A} is an alkali metal.

14. The polymer of claim 8, wherein R⁴ has the following structure: 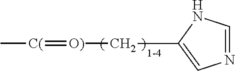
at a pH≧the transition point.

15. The polymer of claim 8, wherein R⁴ has the following structure: 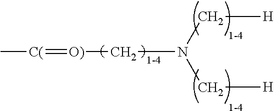
at a pH≧the transition point.

16. The polymer of claim 8, wherein R⁴ has the following structure: 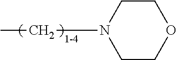
at a pH≧the transition point.

17. The polymer of claim 8, wherein R⁴ has the following structure: 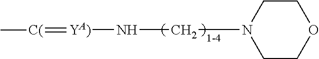
at a pH≧the transition point, wherein Y^{A} is S or O.

18. The polymer of claim 8, wherein the transition point is at a pH<7.4.

19. The polymer of claim 8, wherein the transition point is at a pH<5.

20. The polymer of claim 8, wherein an amount of the polymer is more insoluble in a solvent at a pH less than the transition point compared to the same amount of the same polymer in the same solvent at a pH greater than or equal to the transition point.

21. The polymer of claim 8, further comprising a nucleic acid associated with the polymer, wherein the nucleic acid is selected from the group consisting of DNA, RNA siRNA and antisense.

22. A pharmaceutical composition comprising the polymer of claim 1 and a pharmaceutically acceptable carrier.

23. A method for transfecting a cell comprising delivering to the cell the polymer of claim 21.

24. A method for treating a tumor comprising administering an effective amount of the polymer of claim 21 to a subject with the tumor.

25. A method for treating a tumor comprising contacting a tumor cell with an effective amount of the polymer of claim 21 to a subject with the tumor.
